(19) United States
(12) Patent Application Publication
(10) Pub. No.: US 2026/0234014 A1
(43) Pub. Date: Aug. 13, 2026

(54) PROCESS FOR MANUFACTURING HIGH-PURITY MAGNESIUM OXIDE FROM MAGNESIUM CHLORIDE

(71) Applicant: Purac Biochem B.V., Gorinchem (NL)

(72) Inventors: Fesia Lestari LAKSMANA, Gorinchem (NL); Gerard Hendrik VAN BOCHOVE, Gorinchem (NL)

(21) Appl. No.: 19/649,943

(22) Filed: Apr. 16, 2026

Related U.S. Application Data
(63) Continuation of application No. PCT/EP2024/079103, filed on Oct. 16, 2024.

(30) Foreign Application Priority Data
Oct. 17, 2023 (EP) 23204094.9

(51) Int. Cl. C01F 5/10; C12P 7/40

(52) U.S. Cl. CPC C01F 5/10; C12P 7/40

(57) ABSTRACT

Magnesium chloride is converted to high-purity magnesium oxide and HCl by feeding an aqueous magnesium chloride solution to a spray dryer operated as a thermohydrolysis reactor at a temperature of at least 300° C. Heat for the reactor is generated by burning a combustible gas that contains at least 30 mole % hydrogen, based on total moles of combustible compounds in the gas. A solid magnesium oxide product and an HCl-containing gas stream are withdrawn from the reactor. The solid product contains at least 97.5 wt. % MgO and less than 2.5 wt. % total magnesium hydroxychloride and magnesium chloride, based on total MgO, magnesium hydroxychloride, and magnesium chloride. The process enables production of high-purity magnesium oxide at relatively low spray-dryer operating temperatures. Also disclosed is a carboxylic acid manufacturing process in which magnesium chloride is processed to regenerate magnesium oxide and HCl.

### CROSS REFERENCE TO RELATED APPLICATIONS

The present application is a continuation of International Application No. PCT/EP2024/079103, filed Oct. 16, 2024, which claims priority to European Patent Application No. 23204094.9, filed Oct. 17, 2023, both of which are hereby incorporated by reference in their entireties.

### BACKGROUND

The present invention pertains to a process for manufacturing high-purity magnesium oxide from magnesium chloride solutions. The invention also pertains to a process for manufacturing a carboxylic acid through fermentation, in which the process for manufacturing high-purity magnesium oxide from magnesium chloride solutions has been integrated.

Methods for manufacturing carboxylic acids through fermentation are known in the art. In these methods, a base is often added to keep the pH of the fermentation medium at the desired value. This results in the carboxylic acid being obtained in the form of a salt, e.g. a magnesium salt. Upon recovering the carboxylic acid, salt solutions, e.g., magnesium salt solutions are obtained, which require further processing. Preferably, the salt solutions are processed to form materials which are suitable for recycling to earlier steps of the fermentation. In particular, it is preferred to process the salt solution to form a base that can be used for pH control in the fermentation.

WO00/17378 describes a method for manufacturing lactic acid, wherein in a fermentation process a magnesium lactate solution is prepared. The magnesium lactate solution is acidified with HCl to yield a solution comprising lactic acid in a magnesium chloride solution. The lactic acid is recovered from the solution. The resulting magnesium chloride solution may be processed by subjecting it to a thermohydrolysis step at a temperature of at least 500° C. to react the magnesium chloride with water to yield magnesium oxide powder and hydrochloric acid. The heat required for the thermohydrolytic reaction is provided by the in situ combustion of fuel. WO2013/025106 describes a method for manufacturing carboxylic acids through a process comprising the steps of acidifying a magnesium salt of a carboxylic acid with HCl to form an acid and a magnesium chloride solution, and isolating the acid from the solution through precipitation. It is indicated that the magnesium chloride solution may be processed through thermal decomposition.

WO2013/093028 describes a method for manufacturing carboxylic acids through a process comprising the steps of acidifying a magnesium salt of a carboxylic acid with HCl to form an acid and a magnesium chloride solution, and isolating the acid from the solution through extraction followed by back extraction. It is indicated that the magnesium chloride solution may be processed through thermal decomposition.

In the references cited above, the magnesium chloride solutions are processed by providing the solution to a thermal decomposition step, where the magnesium chloride reacts with water from the solution to form solid magnesium oxide and a gas stream comprising water and HCl.

A problem with the thermal decomposition processes described in these references is that the magnesium oxide recovered from the thermal decomposition step may be of insufficient purity to recycle it to the fermentation process, either directly or after conversion into magnesium hydroxide. More specifically, it has been found that the chloride content of the magnesium oxide content may be so high that the chloride detrimentally affects the fermentation process. Additionally, high chloride contents may place additional requirements on the reaction vessel, e.g., as regards corrosion resistance.

WO2021/214025 addresses this problem by the provision of a two-step process, in which in a first step a magnesium chloride solution is spray-dried to form a product comprising 10-80 wt. % magnesium oxide and 20-90 wt. % of the total of magnesium hydroxychloride and magnesium chloride, and in a second step the spray-dried product is subjected to a roasting step in a roaster at a temperature of 600-900° C. in the presence of water, resulting in the formation of a product comprising at least 98 wt. % of MgO, and less than 2 wt. % of the total of magnesium hydroxychloride and magnesium chloride. While the process of WO2021/214025 makes it possible to obtain a magnesium oxide product with a chloride content which is so low that it can be recycled to the fermentation step, the fact that it is a two-step process requiring both a spay dryer and a separate roaster may make it less attractive from an apparatus investment point of view. There is therefore need in the art for a process for converting magnesium chloride into magnesium oxide which yields a high-purity magnesium oxide product in a simple and cost-effective manner. The present invention provides such a process.

### SUMMARY OF THE INVENTION

The present invention pertains to a process for converting magnesium chloride into magnesium oxide and HCl comprising the steps of
subjecting a magnesium chloride solution with a magnesium chloride concentration of 30-50 wt. % to thermohydrolysis in a thermohydrolysis reactor which is a spray dryer, the reactor being at a temperature of at least 300° C., the heat in the thermohydrolysis reactor being generated through the combustion of combustible gas which is provided through a burner,
withdrawing a solid magnesium oxide product and a HCl containing gas stream having a temperature of at least 300° C. from the thermohydrolysis reactor,
characterised in that the combustible gas comprises at least 30 mole % of H₂, calculated on the total moles of combustible compounds in the combustible gas, and in that the solid magnesium oxide product has an MgO content of at least 97.5 wt. % and less than 2.5 wt. % of the total of magnesium hydroxychloride and magnesium chloride, calculated on the total of these three components. Less than 2.5 wt. % of the total of magnesium hydroxychloride and magnesium chloride, calculated on the total of MgO, magnesium hydroxychloride and magnesium chloride, corresponds to a chloride content of less than 1.2 wt. %.

Surprisingly it has been found that the use of hydrogen as combustible compound results in the formation of a high purity magnesium oxide product, while the processing temperatures in the spray dryer can be kept relatively low at the same time. The latter is attractive from an economic point of view. Further advantages from the present invention and specific embodiments thereof will become apparent from the further specification.

It is noted that WO2021/214025 mentions the possibility of using hydrogen as combustible gas, in a list with natural gas, biogas, and syngas. The reference does not recognise that the use of hydrogen gas would allow single-step operation to obtain a magnesium oxide product with a low chloride content rather than the two-step process of WO2021/214025 in which a spray-drying step with limited conversion is followed by a high-temperature roasting step.

The invention will be discussed in more detail below.

### DETAILED DESCRIPTION

In the following, reference will be made to the following figures, without being limited thereto or thereby.

### BRIEF DESCRIPTION OF THE DRAWINGS

FIG. 1 illustrates a first embodiment of the process according to the invention.

FIG. 2 illustrates a further embodiment of the process according to the invention.

FIG. 3 illustrates the integration of the process according to the invention into the manufacture of carboxylic acid.

In the process according to the invention a magnesium chloride solution with a magnesium chloride concentration of 30-50 wt. % is provided to a thermohydrolysis reactor. If the magnesium chloride concentration is low, the amount of water to be evaporated in the spray drying step will be higher, making the process less attractive from an energy point of view. On the other hand, where the magnesium chloride concentration is too high, there is a risk of precipitation of magnesium chloride in the spray drying nozzle. In general, it is preferred for the magnesium chloride solution to have a concentration which is as high as possible, without precipitation of magnesium chloride in the nozzle occurring. Therefore, it is preferred for the magnesium chloride solution to have a magnesium chloride concentration in the range of 30-48 wt. %, more in particular in the range of 35-47 wt. %.

It is preferred for the magnesium chloride solution used in the process according to the invention to contain only limited amounts of other compounds than magnesium chloride and water, as the aim is to prepare magnesium oxide with high purity. More in particular, it is preferred that the total of magnesium chloride and water makes up at least 95 wt. % of the magnesium chloride solution, in particular at least 98 wt. %, more in particular at least 99 wt. %.

The temperature of the magnesium chloride solution as provided to the spray-drying step is not critical, and may vary between room temperature and 140° C. Higher temperatures and more concentrated magnesium chloride solutions are preferred, because this results in less energy consumption in the spray drying step. Higher temperatures can be obtained, e.g., by heating the solution in a heat exchange step using hot gas or hot liquid, in manners known in the art. It may be preferred for the magnesium chloride solution as provided to the spray-drying step to have a temperature in the range of 50-140° C., in particular in the range of 70-135° C., more in particular in the range of 90-130° C.

The magnesium chloride solution is provided to a thermohydrolysis reactor in the form of a spray dryer. Spray-drying processes and apparatus are known in the art. In a spray drying apparatus, the feed to be spray dried is provided to a spray tower, and sprayed through a nozzle to form small droplets. The droplets fall down through hot gas, and solidify in the process under the evaporation of water. In the process of the invention, magnesium chloride is converted into magnesium oxide during the spray-drying.

In the process according to the invention, the spray-dying step is carried out at a temperature of at least 300° C. The temperature is the temperature of the gas at the location of the spray-drying nozzle, i.e., in the top of the unit. It is preferred for the temperature to be at most 600° C., in particular at most 550° C. as higher temperatures are associated with higher energy consumption, while not being required to obtain the desired degree of conversion. The process according to the invention may be carried out at lower temperatures also, e.g., at most 500° C., or even at most 480° C. Lower temperatures have been found preferrable for reasons of energy conservation and to help prevent the formation of NOx. To ensure sufficient conversion, it is preferred for the spray drying temperature to be at least 350° C., in particular at least 375° C., more in particular at least 400° C.

The residence time in the spray-drying apparatus, defined as the time between the moment that the droplet leaves the nozzle and the moment that the solid particles reach the bottom of the unit, generally is in the range of 1 to 90 seconds, in particular in the range of 3 to 60 seconds, e.g., 5-45 seconds or 5-30 seconds. The residence time is governed by the droplet/particle size, height of the spray tower and the flow velocity and direction of any gas streams provided to the spray tower.

The residence time and temperature have effect on the extent of conversion that will be obtained. Higher temperatures and longer residence times will result in higher degrees of conversion. It is within the scope of the skilled person to determine the residence time and temperature required to achieve the desired degree of conversion based on his common general knowledge and the teachings of the present specification.

In the spray-drying step, gas is provided to the unit to provide the necessary temperature and airflow. In the process of the present specification, the heat in the thermohydrolysis reactor is generated through the combustion of combustible gas in a burner, wherein the combustible gas comprises at least 30 mole % of H₂, calculated on the total moles of combustible compounds in the combustible gas. As indicated above, it has been found that the use of hydrogen as combustible compound results in an increased conversion of magnesium chloride into magnesium oxide as compared to a process carried out at the same temperature but using natural gas as combustible gas. It is therefore preferred for the combustible gas to comprises at least 40 mole % of H₂, calculated on the total moles of combustible compounds in the combustible gas, in particular at least 50 mole %, more in particular at least 60 mole %.

In one embodiment, the combustible gas comprises at least 70 mol. % of H₂, in particular at least 80 mole % of H₂, or at least 90 mole % calculated on the total moles of combustible compounds in the combustible gas. It may be preferred for the combustible gas to comprises at least 95 mole % of H₂, calculated on the total moles of combustible compounds in the combustible gas. In another embodiment, depending on the availability of hydrogen and other combustible gases and on the desired degree of conversion, the combustible gas may contain other combustible compounds, as long as the percentages above are taken into account. Therefore, in one embodiment, the combustible gas comprises 60 to 95 mol. % hydrogen and 5 to 40 mol. % of other combustible compounds, calculated on the total moles of combustible compounds in the combustible gas, e.g., 60-80 mol. % of H₂ and 20-40 mol. % of other combustible compounds. Examples of sources of other combustible compounds are natural gas and biogas. These gases contain methane as major combustible compound (generally present in an amount of at least 70 wt. %, calculated on the total amount of combustible compound in those gases, in particular at least 80 wt. %, in some embodiments at least 90 wt. %), while other combustible compounds, in particular C2, C3, and C4 alkanes, may also be present.

Further gaseous components will also be provided to the burner unit. Obviously, oxygen will be provided to ensure combustion of the combustible gases. The required amount of oxygen can be calculated from the amount of combustible compounds. In general, the amount of oxygen will be in the range of 1-2 times the stoichiometric amount of oxygen required fully convert the combustible compounds into H₂O and, where applicable CO₂. It may be preferred for the amount of oxygen to be provided to be 1.05-1.5 times the amount required to fully convert the combustible compounds into H₂O and, where applicable CO₂, more in particular 1.1-1.3 times. Preventing too large excess of oxygen has been found to help prevent the formation of NOx.

In addition to oxygen and combustible gas, inert gas will also be provided. This is necessary to ensure an adequate gas flow in the unit, and to prevent overheating. Inert gas within the meaning of the present specification will encompass all gases which are not combustible and which are not oxygen. Examples of inert gas that may be provided to the burner include nitrogen, carbon dioxide, and water and combinations thereof. It is preferred that the total of nitrogen, carbon dioxide, and water make up at least 90 wt. % of the inert gas provided to the burner, in particular at least 95 wt. %, more in particular at least 98 wt. %.

In general, oxygen is provided by the provision of air. This already results in the provision of a substantial amount of inert gas to the unit, as the oxygen content of air is 21 vol. % oxygen and 78 vol. % nitrogen (and other compounds such as noble gases and CO₂ in minor amounts).

The composition of the total gas feed provided to the burner may, e.g., be as follows (all percentages being calculated on the weight of the total gas feed provided to the burner):

The total amount of combustible compounds may be in the range of 0.5-10 wt. %, in particular in the range of 1-5 wt. %. The exact amount of combustible compounds will also depend on the nature of the compounds.

Hydrogen may, e.g., be present in an amount of at least 0.1 wt. %, e.g., in an amount of at least 0.2 wt. %, or at least 0.3 wt. %. Depending on unit operations, and in particular on the amount of other combustible compounds, 5 wt. % or 3 wt. % may be mentioned as upper limits of hydrogen gas in the total gas feed to the burner.

Oxygen may, e.g., be present in an amount of 18-25 wt. %, e.g., 20-23 wt. %.

Inert gas may, e.g., be present in an amount of 70-85 wt. %. Nitrogen is particularly suitable as inert gas, in view of its inertness and its abundant availability in e.g., air. It may therefore be preferred for the inert gas to consist for at least 90 wt. %, in particular 94 wt. %, more in particular at least 96 wt. % of nitrogen calculated on the weight of the inert gas.

In one embodiment, the gas feed to the burner is provided by providing a combustibles-containing gas stream, an oxygen-containing gas stream, which in particular may be an air stream, and a diluting gas stream. The various streams can be premixed before being provided to the burner, but they may also be provided to the burner separately. Premixing some streams and separate provision of other streams is also possible. Premixing the combustibles-containing gas stream and the oxygen-containing gas stream is to be avoided, as in that case combustion will start in the absence of the diluting gas, therewith negating its purpose.

The combustibles-containing gas stream generally comprises combustible compounds in an amount of at least 50 wt. %, calculated on the weight of this combustibles-containing stream, in particular at least 60 wt. %, more in particular at least 70 wt. %, still more in particular at least 80 wt. %, even more in particular at least 90 wt. %. The remainder of the combustibles-containing gas stream will be inert gas.

The oxygen-containing gas stream will generally be air, although other oxygen-containing gas streams may also be used. In general, the oxygen-containing gas stream will comprise between 5 and 30 wt. % oxygen, in particular between 15 and 25 wt. % oxygen calculated on the weight of this oxygen-containing gas stream. The remainder of the oxygen-containing gas stream will be inert gas.

The use of a diluting gas stream may be attractive because it makes it possible to dilute oxygen, thus resulting in improved temperature control and reduced NOx formation in particular in reaction with hydrogen. In one embodiment, the diluting gas stream contains at most 1 wt. % of combustible compounds, in particular at most 0.5 wt. %, more in particular at most 0.2 wt. %, even more in particular at most 0.1 wt. % calculated on the weight of this diluting gas stream. The diluting gas stream contains less than 7 wt. % oxygen, in particular less than 5 wt. % oxygen, more in particular less than 4 wt. % oxygen calculated on the weight of this diluting gas stream. The diluting gas stream will generally comprise nitrogen, generally in an amount of 60-95 wt. %, in particular 65-80 wt. %. Depending on its source, the diluting gas stream may contain carbon dioxide, e.g., in an amount up to 15 wt. %. Depending on its source, it may contain water, e.g., in an amount up to 30 wt. %, e.g., in the range of 5-25 wt. %.

The relative amounts of the various gas streams can be selected to obtain the desired amounts of the various components to be provided to the burner, as described above.

The provision of a diluting gas stream may be a way to provide an inert cooling means to the system, to help to address possible temperature peaks. Accordingly, in one embodiment, the diluting gas stream has a temperature of at most 150° C., in particular at most 100° C., more in particular at most 80° C., more in particular at most 60° C. Provision of a diluting gas at low temperature has been found to be an attractive means of reducing NOx formation.

The amount of the diluting gas stream will be determined by the composition of the various gas streams, and by the desired total composition of the gas to be provided to the burner.

If a diluting gas stream is provided, it may be preferred to provide it in an amount in the range of 1-10 kg diluting gas per kg combustible gas, e.g., in the range of 1-8 kg/kg, specifically in a range of 2-6 kg/kg.

Gas is also withdrawn from the spray drying unit. The gas that is withdrawn from the unit contains HCl generated by the decomposition of the magnesium chloride into magnesium oxide. The gas that is withdrawn from the unit also contains water evaporated from the magnesium chloride solution. The HCl concentration in this gas stream generally is in the range of 5-20 wt. %, in particular 7-17 wt. %, e.g., 10-15 wt. %. The HCl-containing gas stream generally comprises 20-50 wt. % of water, in particular 23-45 wt. %, e.g., 25-35 wt. %. Its oxygen content is generally less than 10 wt. %, in particular less than 7 wt. %. Depending on the further composition, the HCl-containing gas stream generally comprises at least 25 wt. % of gases selected from the group consisting of N₂, CO₂ and mixtures thereof. The inert gas concentration may be higher, e.g., at least 50 wt. In one embodiment, the gas feed may comprise 40-80 wt. % nitrogen gas.

The gas stream withdrawn from the spray drying unit may further comprise MgO particles entrained in the upward flow of gas in the unit.

The temperature of the gas withdrawn from the unit at its withdrawal point is in the range of temperatures given for the thermohydrolysis temperature (which is determined at the location of the spray nozzle at the top of the unit), i.e., at least 300° C. The temperature preferences expressed above for the thermohydrolysis reactor also apply here.

The gas stream can be treated as desired.

Solid MgO particles, if present, can be removed from the gas stream by providing the gas stream to suitable separation apparatus. For example, a cyclone may be used, but other apparatus such as filters, precipitators or scrubbers may also be used. The MgO particles recovered from the gas stream may be processed as desired. They can, e.g., be provided to the spray dryer, but they can also be combined with the MgO withdrawn from the spray dryer, or disposed of separately.

HCl can be removed from the gas stream by passing it through an absorber, e.g., an aqueous solution which will absorb HCl from the system to form an aqueous HCl solution.

Heat may be recovered from the gas stream. This can be done, e.g., by contacting the HCl-containing gas stream with a cooling liquid in a cooling step, followed by withdrawing a HCl-containing gas stream with a temperature below 150° C. from the cooling step. The cooling liquid can be used directly to provide heat to streams to be heated, or it can be provided to a heat exchanger, where heat from the cooling liquid is transferred to a heating liquid which circulates from the heat exchanger to a step where heat is utilised. This may, e.g., be a concentration step where water is evaporated from a magnesium chloride solution to increase the magnesium chloride concentration therein.

After removal of HCl and, where applicable, removal of MgO, and cooling, a gas stream is obtained which may be suitable for use as a diluting gas stream as describe above, to be combined with a combustibles-containing gas stream and an oxygen containing gas stream.

The spray-drying step generates solid particles of a magnesium oxide product which comprises at least 97.5 wt. % of MgO, and less than 2.5% wt of the total of magnesium hydroxychloride and magnesium chloride, calculated on the total of these three components. This corresponds to a total chloride content of less than 1.2 wt. %.

The content of the various components may be determined as follows:

The total chloride is determined on a sample of magnesium oxide product dissolved in 70% wt nitric acid by known methods such as chloride titration (using silver nitrate) or XRF or other chloride analytical methods.

Insoluble chloride is determined by washing a sample of magnesium oxide product in excess methanol, where the soluble chloride (i.e. magnesium chloride) will be removed. The chloride content of the insoluble part (i.e. magnesium hydoxychloride) can be determined as above. The soluble chloride is calculated from the total chloride subtracted with the insoluble chloride. The equivalent magnesium chloride and magnesium hydroxychloride is calculated to correspond to the soluble and insoluble chloride, respectively. Total Mg in the samples is determined by titrating a sample of magnesium oxide product (pre-dissolved in 70% wt nitric acid) with ethylene diamine tetraacetic acid tetra sodium salt in ammonium buffer solution. MgO is calculated based on the Mg derived from the total Mg subtracted with the sum of Mg in magnesium chloride and magnesium hydroxychloride.

The process according to the invention makes it possible to obtain high purity products, with an MgO content of at least 97.5 wt. %, in particular at least 98 wt. %, more in particular at least 98.5 wt. %, in some embodiments at least 99 wt. %, and a total of magnesium chloride and magnesium hydroxychloride content of less than 2.5 wt. %, in particular less than 2 wt. %, more in particular less than 1.5 wt. %, in some embodiments less than 1 wt. %.

Depending on the composition of the starting material, the solid product may contain further contaminants. It is, however, preferred for the total of MgO, magnesium hydroxychloride and magnesium chloride to make up at least 95 wt. % of the solid product, in particular at least 98 wt. %, more in particular at least 99 wt. %.

The process according to the invention has been found to result in magnesium oxide product particles having a relatively narrow particle size distribution. The particle size distribution can be characterised by the D-values D10, D50, and D90. D10 is the diameter at which 10% of the mass of a sample is comprised of particles with a diameter below this value. The D50 is the diameter at which 50% of the sample mass has a diameter below this value and 50% of the sample mass has a diameter above this value. D90 is the diameter at which 90% of the sample mass has a diameter below this value. The D-values can be determined by methods known in the art, e.g., laser diffraction on a dispersion of the product in a saturated MgO solution. In one embodiment, the particles obtained by the process according to the invention have a D50 in the range of 0.5 to 20 micron, in particular in the range of 2 to 10 micron.

In one embodiment, the particles have a surface area of 3-20 m²/g, in particular 5-10 m²/g.

The magnesium oxide obtained by the method according to the invention has a high reactivity, as can be quantified by the citric acid reactivity. More in particular, the magnesium oxide has a citric acid reactivity in the range of 75-400 s, preferably 100-375 s, and specifically 125-350 s, or 125-300 s. Citric acid reactivity is determined as follows: An (0.4 eq/L/25.61 g/L) citric acid solution is prepared containing phenolphthalein (30 mg) as indicator. The acid solution was shaken for at least one hour at 30° C. 1±0.01 g of the powdered MgO sample was transferred into 50 ml of the acid solution at 30° C. and shaken until the colour of the slurry changed from white to pink. The time taken for the slurry to change the colour is the citric acid reactivity. The method is analogous to that described in E. M. van der Merwe, Hydration of medium reactive industrial magnesium oxide with magnesium acetate, thermogravimetric study, Journal of Thermal Analysis and calorimetry, Vol 77 (2004) 49-56. It has been found that the reactivity of the magnesium oxide obtained by the process of the invention is slightly higher than that of the magnesium oxide obtained by a process with an additional roaster after the spray drying step at the same chloride content.

Spray-drying apparatus is known in the art, and requires no further elucidation here.

The process according to the invention yields a magnesium oxide with high purity and low chloride content, which is suitable for use as neutralization agent in a fermentation process for the manufacture of carboxylic acids. The invention thus also pertains to a process for manufacturing carboxylic acids through fermentation employing a magnesium oxide obtained through the process according to the invention.

The invention also pertains to an integrated process comprising a fermentation step, a separation step, and a step for converting a magnesium chloride solution into high-purity magnesium oxide. This embodiment will be discussed in more detail below.

In one embodiment, the invention provides a process for the manufacture of carboxylic acid comprising the steps of
subjecting a carbon source to a fermentation step to form a carboxylic acid, which fermentation step comprises the steps of fermenting a carbon source by means of a micro-organism in a fermentation broth to form carboxylic acid and neutralizing at least part of the carboxylic acid by adding a magnesium base selected from magnesium oxide and magnesium hydroxide, thereby obtaining a magnesium carboxylate,
subjecting the magnesium carboxylate to an acidification step wherein the magnesium carboxylate is contacted with HCl in an aqueous environment to form an aqueous mixture comprising carboxylic acid and magnesium chloride,
subjecting the aqueous mixture comprising carboxylic acid and magnesium chloride to a separation step, to form an effluent comprising carboxylic acid and an aqueous magnesium chloride solution,
providing an magnesium chloride solution with a magnesium chloride concentration of 30-50 wt. %,
subjecting the magnesium chloride solution with a magnesium chloride concentration of 30-50 wt. % to thermohydrolysis in a thermohydrolysis reactor which is a spray dryer, the reactor being at a temperature of at least 300° C., the heat in the thermohydrolysis reactor being generated through the combustion of combustible gas which is provided through a burner, wherein the combustible gas comprises at least 30 mole % of H₂, calculated on the total moles of combustible compounds in the combustible gas,
withdrawing a solid magnesium oxide product and a HCl containing gas stream having a temperature of at least 300° C. from the thermohydrolysis reactor, the solid magnesium oxide product having an MgO content of at least 97.5 wt. % and less than 2.5% wt of the total of magnesium hydroxychloride and magnesium chloride (equivalent to 1.2% wt chloride), calculated on the total of these three components.

In one embodiment, solid magnesium oxide product generated by the thermohydrolysis step is provided as neutralising agent to the fermentation step. It can be used as such. It can also be used after conversion into magnesium hydroxide by reaction with water. The step of converting magnesium oxide into magnesium hydroxide through reaction with water can be carried out by methods known in the art. The amount of magnesium base added during the fermentation step generally is in the range of 0.1 to 4 mole magnesium base per mole carboxylic acid, more specifically in the range of 0.2 to 2 mole magnesium base per mole carboxylic acid.

Depending on its concentration, the magnesium chloride solution obtained from the separation step can be provided directly to the thermohydrolysis step. Generally, however, the aqueous magnesium chloride solution derived from the separation step will have a relatively low concentration. Therefore, it may be required to subject the aqueous magnesium chloride solution derived from the separation step to one or more concentration steps where water is evaporated. This results in a more concentrated solution, which is then provided to the spray-drying step. In one embodiment an aqueous magnesium chloride solution with a magnesium chloride concentration of 10-30 wt. % is subjected to a concentration step where water is evaporated, resulting in a concentrated magnesium chloride solution with a magnesium chloride concentration of 30-50 wt. %, in particular 30-48 wt. %, in particular 35 to 47 wt. %, which is then provided to the spray-drying step. The concentration step or steps can be carried out by methods known in the art, which require no further elucidation here.

As discussed above, a HCl-containing gas stream is recovered from the spray-drying step. In one embodiment, at least part of the HCl derived this step is provided to the acidification step, wherein magnesium carboxylate is contacted with HCl in an aqueous environment to form an aqueous mixture comprising carboxylic acid and magnesium chloride. Depending on the process, it is possible to provide the HCl to the acidification step in gaseous form. Nevertheless, it is generally preferred, to absorb HCl from the HCl containing gas stream into water to form an aqueous HCl solution, and to provide the aqueous HCl solution to the acidification step.

As discussed above, after removal of HCl a gas stream is obtained which can be used as diluting gas stream into the burner at the thermohydrolysis step.

The various steps in the integrated process which are additional to the processing of the magnesium chloride solution will be discussed below.

In the first step a carbon source is subjected to a fermentation step to form a carboxylic acid, which fermentation step comprises the steps of fermenting a carbon source by means of a micro-organism in a fermentation broth to form carboxylic acid and neutralizing at least part of the carboxylic acid by adding a magnesium base selected from magnesium oxide and magnesium hydroxide, thereby obtaining a magnesium carboxylate.

Fermentation processes for the manufacture of carboxylic acids are known in the art and require no further elucidation here. It is within the scope of the skilled person to select, using his common general knowledge, a suitable fermentation process, depending on the desired acid to be produced, the carbon source and the microorganism available.

The product of the fermentation process is a fermentation broth, which is an aqueous liquid comprising magnesium carboxylate, biomass, and optionally further components such as impurities like are sugars, proteins, and salts.

If so desired, the fermentation broth may be subjected to a biomass removal step, e.g., a filtration step, before further processing. This is generally preferred for improving product quality. Depending on the carboxylic acid produced, another intermediate step may be separation of solid reaction product, e.g., magnesium carboxylate, from the fermentation broth, before, after, or simultaneous with biomass removal, and optionally subjecting the magnesium carboxylate to a washing step.

Depending on the carboxylic acid produced, another intermediate step may be subjecting the fermentation broth to a concentration step to increase the concentration of magnesium carboxylate in the composition before acidification. This step may be carried out before, after, or simultaneous with biomass removal.

Other intermediate steps, e.g., purification steps, may be carried out as desired, as will be evident to the skilled person.

The next step in the integrated process according to the invention is subjecting the magnesium carboxylate to an acidification step, also sometimes indicated as acidulation step, wherein the magnesium carboxylate is contacted with HCl in an aqueous environment to form an aqueous mixture comprising carboxylic acid and magnesium chloride.

There are various ways in which this step can be effected.

The acidification step is typically conducted by bringing the carboxylate salt in contact with an acidic HCl solution. However, in some embodiments it may also be possible to contact the carboxylate salt with gaseous HCl.

The carboxylate salt may be in solid and/or dissolved form. In one embodiment, the carboxylate salt is provided in solid form. In this case, the acidification step is conducted by bringing the carboxylate salt in contact with an acidic solution. The advantage of preparing the aqueous mixture from carboxylate salt in solid form is that very high carboxylic acid concentration can thus be obtained, such as concentration of at least 15 wt. %, in particular at least 25%, up to, e.g. 50 wt. %, or e.g. 40 wt. %.

The carboxylate salt may also be in dissolved form, typically as part of an aqueous solution. In this case, the acidification step can be conducted by bringing the carboxylate salt in contact with an acidic solution or an acidic gas.

The acidification step may also be conducted on a mixture of carboxylic acid and carboxylate salt. Such a mixture may for example be obtained in a low pH fermentation. The mixture may for example be an aqueous suspension.

When acidification of the carboxylate salt is conducted by contacting it with an acidic HCl solution, it preferably has an acid concentration as high as possible. Such a high acid concentration will result in an aqueous mixture with a high carboxylic acid concentration, which is desirable. The acidic solution therefore comprises at least 5 wt. %, more preferably at least 10 wt. % and even more preferably at least 20 wt. % acid, based on the total weight of the acidic solution.

Acidification is typically conducted using an excess of acid. The excess is preferably small, such that the aqueous mixture obtained is not highly acidic, which may not be desirable in view of further processing such a mixture. For example, the excess of acid used may be such that the resulting aqueous mixture has a pH 2 or lower, preferably a pH of 0-1.

In case gaseous HCl is used, it may be contacted by bringing it in contact with a carboxylate solution or suspension. In particular, HCl gas may be blown through the solution or suspension. Preferably, acidification is conducted at a temperature of 75° C. or less. At higher temperatures, it becomes uneconomical to adapt equipment to the harsh conditions of an acidic environment at high temperatures.

The acidification step results in the formation of an aqueous liquid comprising carboxylic acid and magnesium chloride. This aqueous liquid is subjected to a separation step, optionally after intermediate processing steps have been carried out such as a concentration step.

Suitable separation steps are known in the art. The nature of the step to be used depends on the nature and properties of the acids.

Where the carboxylic acid is present in whole or in part as solid in the aqueous liquid, separation can take place using conventional solid-liquid separation methods such as filtration, centrifugation, etc.

Where the carboxylic acid is present in whole or in part as a separate organic phase in the aqueous liquid, separation can take place using conventional liquid-liquid separation methods, e.g., decantation, settling, centrifugation, use of plate separators, use of coalescers, and use of hydrocyclones. An extractant may be added to improve the separation efficiency. Combination of different methods and apparatus may also be used.

Where the carboxylic acid is present dissolved in the aqueous liquid, separation can take place using, e.g., extraction with a suitable extractant.

Where an extractant is present in the process according to the invention, the extractant, which may also be indicated as extraction agent is substantially not miscible with water. The use of an extractant results in the formation of a two-phase system during the separation step which comprises a liquid organic layer comprising extraction agent and carboxylic acid and an aqueous layer comprising dissolved magnesium chloride.

Examples of suitable extractants are aliphatic and aromatic hydrocarbons, such as alkanes and aromatic compounds, ketones, and ethers. Mixtures of various compounds may also be used. Examples of suitable aliphatic alkanes are C5-C10 straight chain, branched, or cyclic alkanes, e.g., octane, hexane, cyclohexane, 2-ethyl-hexane, and heptane.

Examples of suitable aromatic compounds are C6-C10 aromatic compounds, e.g., toluene, xylenes, and ethylbenzene.

Examples of suitable ketones are C5+ ketones, more in particular C5-C8 ketones in the present invention. C5+ stands for ketones with at least 5 carbon atoms. The use of C9+ ketones is less preferred, The use of methyl-isobutyl-ketone (MIBK) has been found to be particularly attractive. Examples of suitable ethers are C3-C6 ethers, e.g., methyl tert-butyl ether (MTBE) and diethyl ether (DEE).

After extraction, the carboxylic acid can be separated from the extractant as desired. In one embodiment this can be done by removing the extractant by evaporation. In another embodiment the carboxylic acid can be recovered from the extractant by an extraction with water or another aqueous liquid.

After separation of the carboxylic acid from the salt, the carboxylic acid can be processed as desired. Examples of further processing steps are purification steps such as one or more of washing, active carbon treatment, recrystallization, distillation, and filtration. Where the carboxylic acid is lactic acid, it can be converted to lactide and PLA.

The nature of the carboxylic acid manufactured is not critical to the integrated process according to the invention.

In one embodiment the carboxylic acid is a mono-, di- or tri-carboxylic acid comprising at least 2, but no more than 8 carbon atoms (C2-C8 carboxylic acid). In one embodiment, the carboxylic acid is selected from the group consisting of lactic acid, succinic acid, propionic acid, 3-hydroxypropionic acid, 2-, 3-, and 4-hydroxybutyric acid, citric acid, fumaric acid, itaconic acid, adipic acid, acrylic acid, levulinic acid, maleic acid, 2,5-furandicarboxylic acid, mandelic acid, malic acid, and tartartic acid. Preferably, the carboxylic acid is selected from the group consisting of lactic acid, succinic acid, propionic acid, 3-hydroxypropionic acid, 2-, 3-, and 4-hydroxybutyric acid and citric acid.

In one embodiment, the carboxylic acid is selected from the mono-carboxylic acids with 2-6 carbon atoms. In one embodiment, the monocarboxylic acid with 2-6 carbon atoms does not contain hydroxyl-groups. Within this group, examples of suitable acids are propionic acid, acrylic acid, butyric acid, and valeric acid.

In another embodiment, the monocarboxylic acid contains at least one hydroxyl-group. Within this group, in one embodiment it may be preferred to select the acid from the group of lactic acid, glycolic acid, 3-hydroxypropionic acid, 2-, 3-, and 4-hydroxybutyric acid. In another embodiment within this group it may be preferred to select the acid from the group of glycolic acid, 3-hydroxypropionic acid, and 2-, 3-, and 4-hydroxybutyric acid. In a further embodiment it may be preferred for the acid to be lactic acid.

In another embodiment, the carboxylic acid is a polycarboxylic acid, more in particular a di- or tri-carboxylic acid comprising at least 2, but no more than 6 carbon atoms (C2-6 carboxylic acid). In one embodiment, the polycarboxylic acid is selected from the group consisting of succinic acid, citric acid, fumaric acid, itaconic acid, adipic acid, maleic acid, 2,5-furandicarboxylic acid, malic acid, and tartartic acid. Preferably, the polycarboxylic acid is selected from the group consisting of succinic acid, citric acid, fumaric acid, itaconic acid, adipic acid, and 2,5-furandicarboxylic acid. The polycarboxylic acid may in particular be selected from succinic acid, fumaric acid, itaconic acid, and 2,5-furandicarboxylic acid.

Various aspects of the present invention will be illustrated by the figures discussed below, the invention not being limited thereto or thereby. The following is noted with respect to the figures:

Embodiments of various figures can be combined unless they are mutually exclusive. The figures are flow sheets illustrating the process according to the invention. The figures do not present a reactor set up. For example, where a separation step is shown in a single step, it may be carried out on more than one reactor. This applies, e.g., to distillation steps and crystallisation steps. Conversely, different steps may be carried out in the same unit. By the same token, the various lines are intended to show how components flow from one reaction step to the other. They do not represent real-life structures. The figures are not intended to show specific engineering features or details, including the design of the various components shown.

The figures do not always show all elements of the process according to the invention.

The figures do not show all purge streams or make-up streams that may be present in the practical performance of the process according to the invention although, as will be evident to the skilled person, purge streams and make-up streams may be necessary in practice to maintain stable operation. In addition, auxiliary equipment such as various valves, (vacuum) pumps, heating and cooling equipment, including inlets and outlets for cooling media, analytical devices, control devices and the like are not always shown in the figures, but of course such equipment can be used as necessary or desirable, and is well known the skilled person.

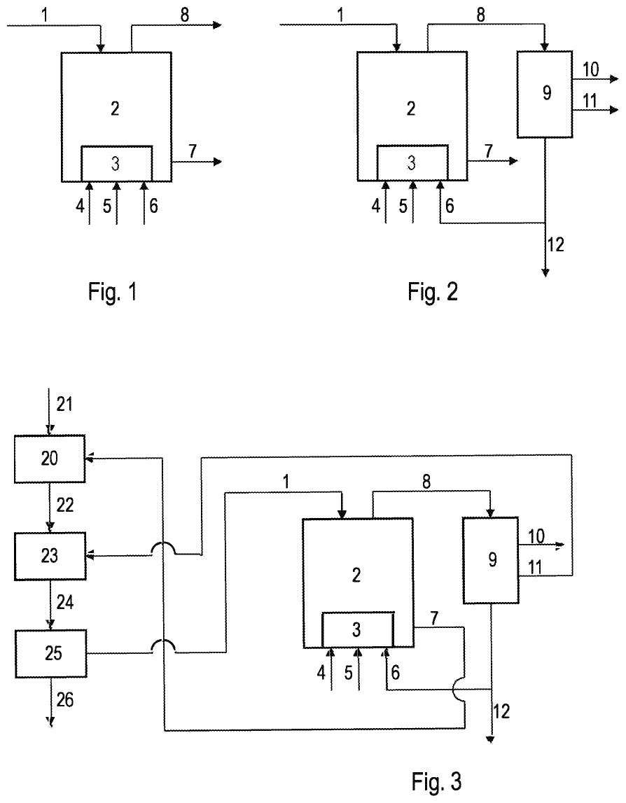

FIG. 1 illustrates the process according to the invention. In FIG. 1, a magnesium chloride solution is provided through line (1) to spray drying apparatus (2). Spray drying apparatus (2) is provided with burner (3), which is provided with, in the illustrated embodiment, three gas feeds, namely, a feed (4) for combustible compounds (including H₂), a feed (5) for oxygen containing gas such as air, and a feed (6) for diluting gas. As explained above, the gas provided to the burner may be mixed before being provided to the burner, but the various streams may also be provided separately, as illustrated here. In the spray drying apparatus (2) the magnesium chloride is converted into a high purity magnesium oxide product, which is withdrawn through line (7). A HCl containing gas stream is withdrawn through line (8).

FIG. 2 is variation on FIG. 1, in which the HCl-containing gas stream withdrawn through line (8) is provided to a separation and cooling step (9). Separation and cooling step (9) is provided here as a single “box” but it will generally consist of a number of connected units. Separation and cooling step (9) generates, in the illustrated embodiment, solid magnesium oxide particles withdrawn through line (10). As explained above, this product can be recycled to the spray drying unit, combined with the magnesium oxide product withdrawn through line (7), or processed separately. Separation and cooling step (9) also generates a HCl-containing aqueous solution, through absorption of the HCl from the HCl-containing gas stream in an aqueous liquid. The HCl-containing aqueous solution is withdrawn through line (11). Separation and cooling step (9) also yield a cooled gas stream, withdrawn through line (12). In the embodiment as illustrated in this figure, part of the cooled gas stream is provided to burner (3) as diluting gas stream.

FIG. 3 shows illustrates a possible way in which the embodiment of FIG. 2 may be integrated into a process for manufacturing carboxylic acid. In FIG. 3, a carbon source to be fermented is provided through line (21) to fermentation step (20). Solid magnesium oxide product is provided as neutralizing agent through line (7). The product from line (10) can be processed in the same way, if so desired. The product may be provided as such, but it may also be converted to magnesium hydroxide (in a step not shown) before providing it to the fermentation step (20). Fermentation step (20) yields a magnesium carboxylate product which is withdrawn through line (22) and provided to acidification step (23). Intermediate steps such as biomass removal or concentration may be carried out, but are not shown. In the acidification step (23) the magnesium carboxylate is contacted with HCl in an aqueous environment to form an aqueous mixture comprising carboxylic acid and magnesium chloride. In the illustrated embodiment, HCl is provided through line (11). Acidification step (23) yields an aqueous mixture comprising carboxylic acid and magnesium chloride, which is provided to a separation step (25) through line (24). The separation step may be carried out as described above. Separation step (25) results in an effluent comprising carboxylic acid and a magnesium chloride solution. The product carboxylic acid is withdrawn through line (26). The magnesium chloride solution is withdrawn through line (1), and provided to the spray-dryer (2), as discussed above in the context of FIG. 1, optionally after passing through an evaporator (not shown) to increase its concentration.

As will be evident to the skilled person, it is possible to provide only part of the HCl generated in the spray-dryer to the acidification step, and/or to also provide HCl from other sources. By the same token, it will be evident that it is possible to provide only part of the magnesium oxide product generated to the fermentation step. Other variations on the processes exemplified in the figures will also be clear to the skilled person.

It will be clear to the skilled person that in the process according to the invention preferred embodiments of various steps can be combined unless they are mutually exclusive.

The present invention is further illustrated by the following examples, without being limited thereto or thereby.

### EXAMPLES

### Example 1—Comparative-Spray Drying Using Natural Gas as Combustible Gas

A 35-40 wt. % aqueous magnesium chloride solution provided to the nozzle of a spray-dryer at a flow of about 92 L/min. Hot gas was provided in the reactor by combusting natural gas with air in a burner directed to the reactor. The spray-drying temperature (at the top of the unit) was controlled at 500° C. The product from this reactor contained about 1.5-2% wt chloride. The gas at the outlet of the reactor contained between 30-50 ppmV NOx (normalised to 3% volume 02). NOx was determined based on the measurement of NO2 and NO concentration in the gas using known sensors e.g. based on ion selective potentiometry principle.

The carry-over of solid magnesium oxide particles over the top of the unit was 32-35 wt. %, calculated on the total of solid magnesium oxide produced.

### Example 2—Use of 100% Hydrogen—with and without Diluting Gas

For a process carried out under the same conditions as Example 1 (35-40 wt. % aqueous magnesium chloride solution, flow of about 92 L/min, gas outlet temperature of 500° C.), with H₂ being used as combustible gas (the processes being matched with the process of Example 1 to provide the same total energy input, the amount of oxygen adapted to the amount of combustible gas), the results provided in the results table below under test A are obtained. It can be seen that the product has a chloride content of 0.35 wt. %, which is much lower than the chloride content of the product of comparative example 1 above (1.5-2 wt. %).

When the process is carried out under the same conditions, except that in addition to the product of the combustion reaction of hydrogen and air a diluting gas stream is provided, a product is obtained with a the same chloride content, but with reduced NOx formation in the gas stream (test B in Table 1 below). The diluting gas stream is a recycle stream derived from the top of the spray dryer after removal of solid particles and HCl. The composition of the total gas feed to the burner is provided below. As appears from the table, the diluting gas was provided in an amount sufficient to reduce the relative weight amount of oxygen and hydrogen in the feed to the burner with about 10%.

### Composition of the Gas Provided to the Burner in Wt. %

| Test | H₂ | O₂ | CO₂ | H₂O | N₂ |
|---|---|---|---|---|---|
| A (without | 3.0 | 22.5 | 0 | 0 | 75 |
| diluting gas) | | | | | |
| B, C (with | 2.8 | 21 | 0 | 0.8 | 75 |
| diluting gas) | | | | | |

Where the process is carried out with the provision of a diluting gas stream as in test B, but at a temperature of 420° C., the chloride content is increased, but NOx formation is further reduced (test C). Due to the lower temperature energy consumption is also reduced. This illustrates how a balance can be found between product properties, energy consumption, and NOx formation.

### Results

| | | | Carry over of | NOx in the gas | |
|---|---|---|---|---|---|
| | | Temperature | particles via the | at the reactor | Chloride |
| | | of the gas at | gas outlet of the | outlet (ppmV | content in |
| | Diluting | the reactor | reactor (% wt to | normalised at | product |
| Test | gas | outlet (° C.) | total particles) | 3% Oxygen) | (% weight) |
| A | 0 | 500 | Not determined | >30 | 0.35 |
| B | yes | 500 | 59 | 19 | 0.35 |
| C | yes | 420 | 48 | 16 | 0.98 |

### Example 3—Use of Gas Mixtures

The process may also be carried out using mixtures of hydrogen with other combustible gases, e.g. biogas. Where a feed is used comprising as combustible compounds 70 vol. % of hydrogen and 30 vol. % methane derived from biogas, the following is obtained. Diluting gas is provided in the same relative amount to the total gas provided to the burner as in Example 2.

| | | NOx in the gas | |
|---|---|---|---|
| | Temperature | at the reactor | Chloride |
| | of the gas at | outlet (ppmV | content in |
| | the reactor | normalised at | product |
| Test | outlet (° C.) | 3% Oxygen) | (% weight) |
| D | 500 | 13 | <1 |
| E | 420 | 10 | <1 |

### Example 4—Temperature of Diluting Gas

It has appeared that the temperature of the diluting gas provided to the unit influences the NOx formation when hydrogen is used as a combustion gas. In both experiments, the products obtained had a chloride content of below 1 wt. %.

| | | | | NOx in the gas |
|---|---|---|---|---|
| | | Temperature | | at the reactor |
| | | of the gas at | Temperature | outlet (ppmV |
| | | the reactor | of the diluting | normalised at |
| Experiment | Fuel | outlet (° C.) | gas (° C.) | 3% Oxygen) |
| X | 100% | 420 | 55 | 20 |
| Y | Hydrogen | 420 | 89 | 25 |

1. A process for converting magnesium chloride into magnesium oxide and HCl, the process comprising:
(a) subjecting a magnesium chloride solution with a magnesium chloride concentration of 30-50 wt. % to thermohydrolysis in a thermohydrolysis reactor that is a spray dryer, the reactor being at a temperature of at least 300° C., the heat in the thermohydrolysis reactor being generated through the combustion of combustible gas provided through a burner; and
(b) withdrawing a solid magnesium oxide product and an HCl-containing gas stream having a temperature of at least 300° C. from the thermohydrolysis reactor;
wherein the combustible gas comprises at least 30 mole % of H₂, based on the total moles of combustible compounds in the combustible gas, and
the solid magnesium oxide product has an MgO content of at least 97.5 wt. % and less than 2.5% wt total magnesium hydroxychloride and magnesium chloride, based on the total of MgO, magnesium hydroxychloride, and magnesium chloride.

2. The process according to claim 1, wherein the combustible gas comprises at least 40 mole % of H₂, based on the total moles of combustible compounds in the combustible gas.

3. The process according to claim 1, wherein the combustible gas comprises at least 50 mole % H₂, based on total moles of combustible compounds in the combustible gas.

4. The process according to claim 1, wherein the combustible gas comprises at least 60 mole % H₂, based on total moles of combustible compounds in the combustible gas.

5. The process according to claim 1, wherein the combustible gas comprises at least 70 mole % H₂, based on total moles of combustible compounds in the combustible gas.

6. The process according to claim 1, wherein the magnesium chloride solution has a magnesium chloride concentration of 30-48 wt. %.

7. The process according to claim 1, wherein the magnesium chloride solution has a magnesium chloride concentration of 35 wt. % to 47 wt. %.

8. The process according to claim 1, wherein the magnesium chloride solution comprises magnesium chloride and water in a combined amount of at least 95 wt. % of the magnesium chloride solution.

9. The process according to claim 1, wherein the temperature at the top of the thermohydrolysis reactor is at most 600° C.

10. The process according to claim 9, wherein the temperature at the top of the thermohydrolysis reactor is from 350° C. to 600° C.

11. The process according to claim 9, wherein the temperature at the top of the thermohydrolysis reactor is from 375° C. to 550° C.

12. The process according to claim 1, wherein a gas feed having the following composition is provided to the burner:
combustible compounds are present in an amount of 0.5-10 wt. %;
hydrogen is present in an amount of 0.1 wt. % to 5 wt. %;
oxygen is present in an amount of 18-25 wt. %; and
inert gas is present in an amount of 70-85 wt. %.

13. The process according to claim 1, wherein oxygen is provided in the form of air.

14. The process according to claim 1, wherein a gas feed provided to the burner comprises a combustibles-containing gas stream, an oxygen-containing gas stream, and optionally a diluting gas stream.

15. The process according to claim 14, wherein the gas feed comprises the diluting gas stream, and wherein the diluting gas stream comprises at most 1 wt. % of combustible compounds, less than 10 wt. % oxygen, and at least 60 wt. % nitrogen, based on the weight of the diluting gas stream.

16. The process according to claim 14, wherein the gas feed comprises the diluting gas stream, and wherein the diluting gas stream is obtained from the HCl-containing gas stream recovered from the thermohydrolysis reactor after the HCl-containing gas has been subjected to at least one separation step to remove HCl, and, if present, solid particulates from the gas stream, and to a cooling step, to reduce the temperature of the HCl-containing gas stream recovered from the thermohydrolysis reactor from a value above 300° C. to a value of at most 150° C.

17. The process according to claim 14, wherein the gas feed comprises the diluting gas stream, and the diluting gas stream has a temperature of at most 150° C.

18. A process for manufacturing a carboxylic acid comprising the steps of:
(a) subjecting a carbon source to a fermentation step to form a carboxylic acid, which fermentation step comprises the steps of fermenting a carbon source by means of a microorganism in a fermentation broth to form carboxylic acid and neutralizing at least part of the carboxylic acid by adding a magnesium base selected from magnesium oxide and magnesium hydroxide, thereby obtaining a magnesium carboxylate,
(b) subjecting the magnesium carboxylate to an acidification step wherein the magnesium carboxylate is contacted with HCl in an aqueous environment to form an aqueous mixture comprising carboxylic acid and magnesium chloride,
(c) subjecting the aqueous mixture comprising carboxylic acid and magnesium chloride to a separation step, to form an effluent comprising carboxylic acid and an aqueous magnesium chloride solution,
(d) recovering the effluent comprising carboxylic acid, and
(e) processing the magnesium chloride to form magnesium oxide and HCl in accordance with the process of claim 1.

19. The process according to claim 18, wherein the magnesium base in the fermentation step comprises a solid magnesium oxide product derived from the thermohydrolysis reactor, directly or after conversion to magnesium hydroxide.

20. The process according to claim 18, wherein HCl derived from the thermohydrolysis reactor is provided to the acidification step.
